(19) United States
(12) Patent Application Publication
(10) Pub. No.: US 2025/0025674 A1
(43) Pub. Date: Jan. 23, 2025

(54) PHARMACEUTICALS CARRIER AND OPERATING METHOD THEREOF

(71) Applicant: NEUCEN BIOMED CO., LTD., Taipei City (TW)

(72) Inventor: MING-FU CHIANG, Taipei City (TW)

(21) Appl. No.: 18/709,471

(22) PCT Filed: Dec. 29, 2021
(86) PCT No.: PCT/CN2021/142435

§ 371 Date: May 10, 2024

(30) Foreign Application Priority Data
Nov. 10, 2021 (CN) 202111323844.6

(51) Int. Cl. A61M 31/00

(52) U.S. Cl. CPC A61M 31/00

(57) ABSTRACT

The present invention discloses a pharmaceuticals carrier and the operating method thereof. The pharmaceuticals carrier comprises a holding portion and an absorbing portion. The holding portion is constructed by two sheets, and the two sheet comprises a first segment, a second segment and an arch portion. The first segment and the second segment are connected, and the absorbing portion is configured on the first segment of the holding portion, for absorbing a medicament.

### BACKGROUND OF THE INVENTION

The present application claims the priority of the China Patent Application filed on Nov. 10, 2021, and China Application Number of the aforementioned priority is 202111323844.6. The disclosure of said priority shall be contained in the present application therein.

### FIELD OF THE INVENTION

The present application is related to a carrier and its operating method. Specifically, the carrier and its operating method are used for preserving a medicament.

### DESCRIPTION OF THE PRIOR ART

In light of the fact that the nasal cavity mucosa of human body is a highly permeable tissue, the drug is usually preferred to use the nasal cavity as a localized or targeted drug administration pathway for treating symptoms such as allergic reactions. For instance, the vasoconstrictors which is used to improve nasal congestion.

However, the commercial drug administrating carrier for nasal cavity is usually designed as an inhalation product (e.g. spray), or drop-in products. The former is usually spray or mask for nasal cavity. However, the spray or mask is disadvantageous for carrying thereon. On the other hand, the latter causes hygiene issue while the over spilled medicament directly dropping on the cloth or floor via gravity.

### SUMMARY OF THE INVENTION

First of all, the present invention discloses a pharmaceuticals carrier. The pharmaceuticals carrier comprises a holding portion and an absorbing portion. The aforementioned holding portion is constructed by two sheets, and further comprises a first segment, a second segment and an arch portion. The first segment is connected with the second segment, and an absorbing portion is configured on the location of the first segment. The absorbing portion absorbs the medicament. Moreover, one of the abovementioned two sheets may selectively and independently be formed by the aforementioned arch portion, or be formed by the first segment, the second segment and the arch portion. The two sheets further connect to another sheet, therefore to form a channel.

Secondly, the present invention further discloses an operating method for using the abovementioned pharmaceuticals carrier. The method comprises that the dry absorbing portion is immersed into a container until the absorbing portion is soaked by the medicament, then the absorbing portion is removed from the container. Thereafter, a hand operates the holding portion, and the absorbing portion which is soaked is entering a nasal cavity. When the medicament is fully smeared on the nasal cavity, the hand operate the holding portion again and pull the absorbing portion out of the nasal cavity.

Specifically, the medicament comprises an allergen and a pharmaceuticals carrier.

The abovementioned brief description of the present invention is intended to provide a basic explanation of several aspects and technical features of the present disclosure. The brief description of the disclosure is not a detailed description of the present disclosure. Therefore, the purpose is not to specifically enumerate the key or important components of the present disclosure, nor is it intended to be used to define the scope of the disclosure and merely represent several concepts of the disclosure in a concise manner.

### BRIEF DESCRIPTION OF THE DRAWINGS

FIG. 1 is a schematic drawing of the pharmaceuticals carrier of an embodiment of the present invention.

FIG. 2 is a schematic drawing of the pharmaceuticals carrier of the other embodiment of the present invention.

FIG. 3 is a schematic drawing illustrating operating method of the pharmaceuticals carrier of an embodiment of the present invention.

FIG. 4 is a schematic drawing illustrating operating method of the pharmaceuticals carrier of another embodiment of the present invention.

FIG. 5 is a schematic drawing illustrating operating method of the pharmaceuticals carrier of the other embodiment of the present invention.

### DETAILED DESCRIPTION OF THE EMBODIMENTS

For understanding the technical features and practical effects of the present invention, implementation can be carried out according to the contents of the detailed description given by way of the preferred embodiments described in detail with reference to the accompanying drawings below.

The at least one embodiment of the present invention is a carrier. Specifically, the carrier is used for preserving a medicament.

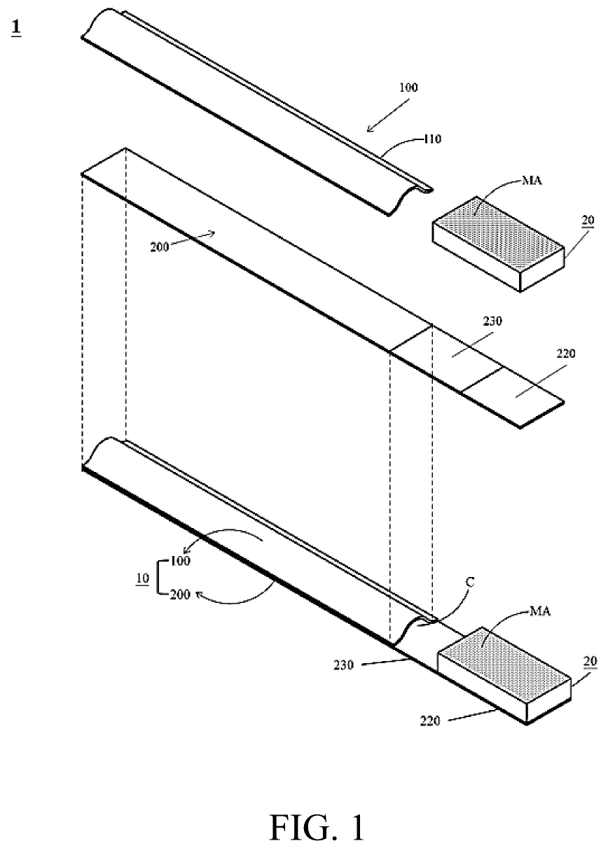

Please refer to FIG. 1, FIG. 1 is a schematic drawing of the pharmaceuticals carrier of an embodiment of the present invention. In FIG. 1, a pharmaceuticals carrier 1 comprises a holding portion 10 and an absorbing portion 20. The holding portion 10 is constructed by two sheets 100 and 200. Specifically, one of the sheets 100 and 200 forms an arch portion 110 relating to the other sheet 100 or 200 along the longitudinal direction of pharmaceuticals carrier 1. The arch portion 110 partially overlaps with the other sheet 200, therefore to form a channel C, a first segment 220 and a second segment 230. The first segment 220 is connected to the second segment 230.

The channel C is directed to an area which the sheet 100 overlaps with sheet 200. Moreover, the first segment 220 and second segment 230 illustrates the non-overlapped area therebetween the sheet 200 and sheet 100. On the other hand, the absorbing portion 20 is configured on the first segment 220 which is on the same side with the aforementioned sheet 200. The absorbing portion 20 soaks and preserves a medicament MA in FIG. 1.

Please notice that the arch portion 110 is an example for describing the configuration per se therebetween the sheet 100 and sheet 200. However, the configuration, order or number of the arch portion 110 may be appropriately changed thereby the material, thickness of sheets 100 and 200. The requirement is that the sectional area of channel C is able to restore the medicament MA therein. For instance, multiple arch portions may be configured on sheet 100 or 200, therefore to match or combine with the other sheet 100 or 200.

Otherwise, the embodiment illustrated in FIG. 1 shows that the cross section of the arch portion 110 is approximately a V shape. However, the cross section of the arch portion 110 is not limited to V shape, the shape of the cross section of the arch portion 110 may be changed with different material, length, width or thickness of sheets 100 and 200, such as the U shape or W shape.

Furthermore, the attached sheets 100 and 200 can be detached in the present embodiment. However, considering to the stability requirement of the channel C, the sheets 100 and 200 may be designed as an unibody structure, therefore to form the aforementioned holding portion 10.

Please refer to the embodiment disclosed in FIG. 1, the sheets 100 and 200 may be manufactured by any suitable materials such as PMMA, PMA, PC, PVC, PDMS, PET, silica gel, rubber or glass thereof. The sheets 100 and 200 may also be designed to show an outer appearance having colors or texture (concave or convex surface), therefore to enhance the ability for user gripping of the holding portion 10.

Furthermore, the absorbing portion 20 may comprise perforated or non-porous non-woven fabrics with absorbable suture in present embodiment. The material of the aforementioned non-woven fabrics may be but not be limited to hydrophilic of hydrophobic man-made fiber (olefin, polyester, polyamide), regenerated fiber comprising Rayon, nanofiber comprising electret material (the electret material may comprise but not be limited to polypropylene, polycarbonate, cyclic olefin copolymer or metallocene cyclic olefin copolymer), and the natural fiber comprising wool, cotton or silk thereof. For instance, the absorbing portion 20 may be the sWet laid Nonwoven which is developed by the Industrial Technology Research Institute (ITRI).

On the other hand, the medicament MA may be a composition which alternatively comprises buffer, adjuvant, excipient or pharmaceutically acceptable carrier. The material of the pharmaceutically acceptable carrier may be selected from but not limited to solvent(s) such as saline, buffered saline, phosphate buffer, borate buffer, glucose solution, oils, and esters thereof. For example, when the aforementioned composition is illustrated to comprise the dust mite extract or the other allergen (e.g. pollen extract, mold extract, animal dander, cockroach extract, food allergen, recombinant allergen peptide or a combination of the above), the pharmaceutically acceptable carrier may be a solvent of glycerin, phenolic compounds, sodium chloride and sodium bicarbonate. Specifically, the pharmaceutically acceptable carrier may be a solvent comprises 50 wt % glycerin, 0.40 wt % phenolic compounds, 0.50 wt % sodium chloride and 0.25 wt % sodium bicarbonate.

When this embodiment is operated, the holding portion 10 may be moved and thereby the absorbing portion 20 is reached into the internal space of nasal cavity. Therefore, all or at least a part of the absorbing portion 20 may be squeezed to contact the inner wall of the nasal cavity, and makes the medicament MA enter and be absorbed by the body of a subject via its mucosa of nasal cavity. Otherwise, the force works on the absorbing portion 20 may be manually and carefully adjusted, therefore to precisely control and operate the contact area between the absorbing portion 20 and the nasal cavity, for controlling the absorbed amount of the medicament MA of the nasal cavity mucosa and preventing the waste of medicament MA.

Moreover, the configuration of channel C sustains the over spilled medicament MA which is not absorbed by the mucosa of nasal cavity. The sustaining mechanism of the channel C therein may be realized by the gravity, adhesion force, surface tension force and cohesion force. Therefore, the medicament Ma may be sustained and prevented to fall on the hand or floor, keeping hygienic and clean when operating the present embodiment.

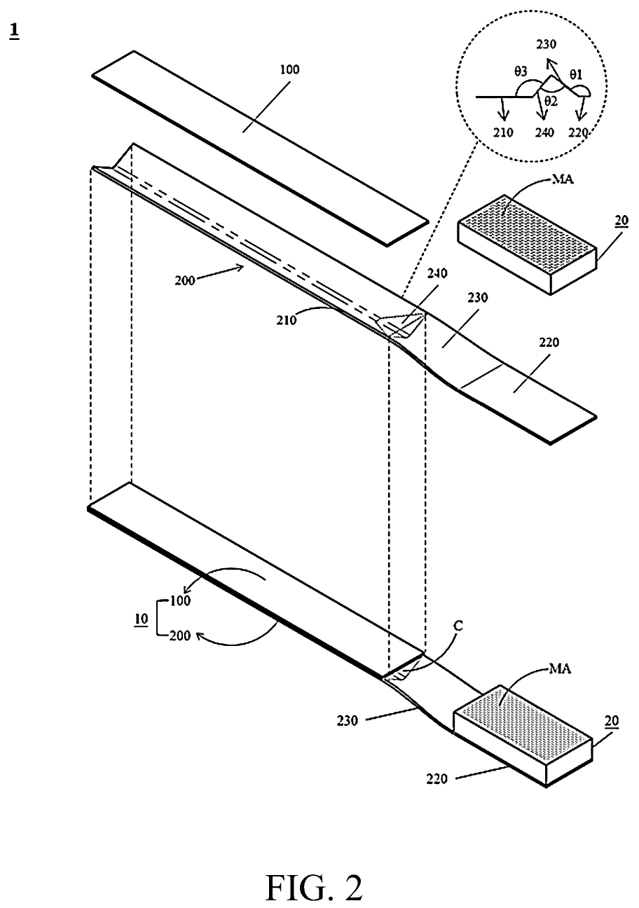

FIG. 2 is a schematic drawing of the pharmaceuticals carrier of the other embodiment of the present invention. The difference between the embodiment of FIG. 1 and the embodiment of FIG. 2 is that the holding portion 10 further comprises a third segment 240. The third segment 240 is extended from the arch portion 210, and the arch portion 210, first segment 220 and second segment 230 form sheets 100 and 200. Specifically, for explaining the configuration of the absorbing portion 20 and holding portion 10, the first angle θ1 is formed between the first segment 220 and second segment 23. The second angle θ2 is formed between the second segment 230 and the third segment 240, and the third angle θ3 is formed by the extension part of third segment 240 which is extended from the arch portion 210. The first angle θ1 is larger or equal to the second angle θ2, and the third angle θ3 is larger or equal to 120 degree but smaller than 180 degree.

In the embodiment disclosed in FIG. 2, the degree of the first angle θ1 and the second angle θ2 will be determined by the force pressed on the absorbing portion 20. The degree of the first angle θ1 and the second angle θ2 may be designed to have a minimum default angle when there is no forces pressed on the absorbing portion 20. The available default angle is determined by the material, length, width and thickness of sheets 100 and 200. For instance, when the sheets 100 and 200 of this embodiment are designed as PET sheets with the length of 50 mm, width of 5 mm, and thickness of 5 mm, the available default angle ranges from 120 to 180 degrees, and the minimum default angle is 135 degrees.

Thereafter, the third angle θ3 can be designed by the material, length, width and thickness of sheets 100 and 200, and the third angle θ3 may also be designed as the same as the minimum default angle of first angle θ1 or the minimum default angle of second angle θ2. For example, when the sheets 100 and 200 of this embodiment are designed as PET sheets with the length of 50 mm, width of 5 mm, and thickness of 5 mm, the minimum default angle of the first angle θ1, the minimum default angle of the second angle θ2 and the minimum default angle of the third angle θ3 are 135 degree.

That is, when the present embodiment is operating, the second segment 230 will be bended and result in the bended first segment 220, because the absorbing portion 20 is configured on the first segment 220. Thereinafter, the contact area between the aforementioned absorbing portion 20 and inner wall of the nasal cavity will be increased while the increase of the pressure, thus to release more medicament MA and raise the absorption possibility of the medicament MA on the mucosa of the nasal cavity in certain period.

On the other hand, the aforementioned structure may prevent the over spilled medicament MA dropping from the first segment 220, the second segment 230 or the third segment 240 when the over spilled medicament MA reaches the channel C via gravity. In fact, the configuration of the first angle θ1, the second angle θ2 and the third angle θ3 may result in that the over spilled medicament MA may naturally passes through the first segment 220, the second segment 230 and third segment 240 via the gravity. Moreover, the flow speed of the over spilled medicament MA may be decreased between the first segment 220 and second segment 230, or be decreased between the second segment 230 and the third segment 240, while the increasing distance which the over spilled medicament MA has been moved. Therefore, the over spilled medicament MA may move in a lower speed, and keep contacting the first segment 220, the second segment 230 or the third segment 240.

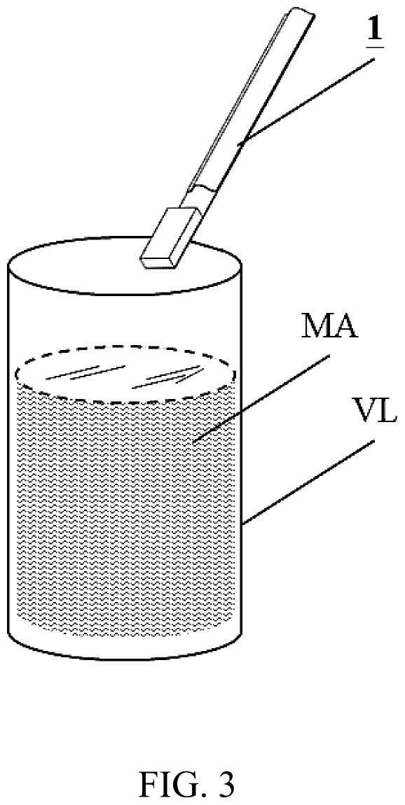

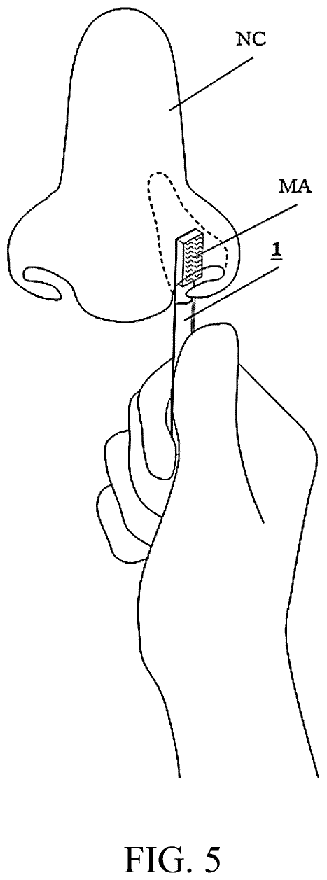

The abovementioned pharmaceuticals carrier(s) 1 are able to be operated under the disclosed method of the present embodiment. Please refer to FIG. 3 to FIG. 5. The FIG. 3 to the FIG. 5 is the operating method of the pharmaceuticals carriers 1 illustrated in FIG. 1 and FIG. 2. As shown in FIG. 3, the dry absorbing portion 20 will be immersed into a container VL which contains the medicament MA before the start of operation, the absorbing portion 20 may be kept in the container VL over 30 seconds, therefore to fully soak the absorbing portion 20 via medicament MA.

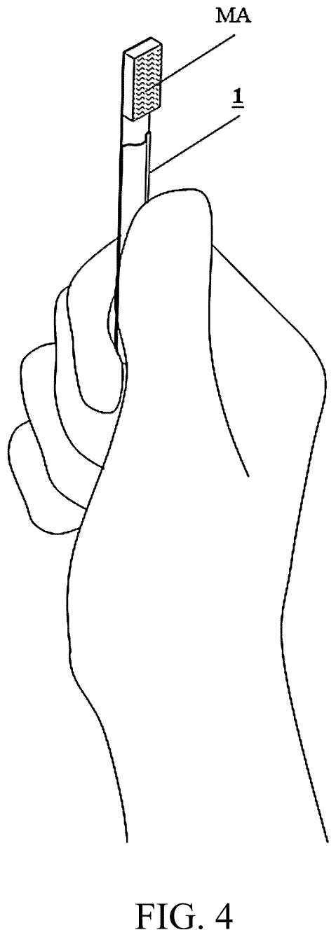

Thereinafter, as shown in FIG. 4 and FIG. 5, the absorbing portion 20 may be pulled out from the container VL via the operation of hand-gripped holding portion 10 before entering the nasal cavity NC. The hand pulls and controls the holding portion 10 and make the absorbing portion 20 enter the nasal cavity NC. Simultaneously, the absorbing portion 2 which absorbs the aforementioned medicament MA will be absorbed by nasal cavity mucosa of human body via the volatilization of the medicament MA per se or the attachment between the absorbing portion 20 and inner wall of the nasal cavity NC. On the other hand, the absorbing portion 20 may be simply pressed or put into the nasal cavity NC over 10 seconds, and the medicament MA will be released, thus to increase the absorption of medicament MA for human body.

The description above provides merely preferred embodiments of the present invention, and is not to be construed as limitations to the scope of implementation of the present invention. All simple and equivalent variations and modifications made to the embodiments based on the claims and the detailed description of the present invention are encompassed within the scope of the present invention.

What is claimed is:

1. A pharmaceuticals carrier, comprising a holding portion and an absorbing portion, wherein the holding portion comprises two sheet, and the two sheets comprises:
a first segment;
a second segment, connected with the first segment; and
an arch portion, configured on one of the two sheets and connected with the second segment;
wherein the absorbing portion is configured on the first segment of the holding portion, and the absorbing portion absorbs a medicament;
wherein the two sheets form a channel via the arch portion.

2. The pharmaceuticals carrier as claimed in claim 1, when one of the two sheets is formed by the first segment, the second segment and the arch portion simultaneously, the holding portion further comprises a third segment; wherein the third segment is extended from the arch portion, and connected with the second segment.

3. The pharmaceuticals carrier as claimed in claim 1, wherein the medicament comprises an allergen and a pharmaceutically acceptable carrier.

4. The pharmaceuticals carrier as claimed in claim 2, wherein the first segment and the second segment form a first angle, and the second segment and the third segment form a second angle; wherein the first angle is larger than or equal to the second angle.

5. The pharmaceuticals carrier as claimed in claim 2, wherein the third segment is extended under a third angle, and the third angle is between 120 degree and 180 degrees.

6. The pharmaceuticals carrier as claimed in claim 3, wherein the allergen is dust mite extract.

7. The pharmaceuticals carrier as claimed in claim 3, wherein the pharmaceutically acceptable carrier comprises solvent of glycerin, phenolic compounds, sodium chloride and sodium bicarbonate.

8. An operating method of the pharmaceuticals carrier as claimed in claim 1, comprising:
immersing the absorbing portion into a container which contains the medicament;
the absorbing portion is removed from the container after the absorbing portion is soaked by the medicament;
the holding portion is operated via a hand, and the absorbing portion which is soaked is put into a nasal cavity;
waiting for the medicament smearing the nasal cavity; and
removing the absorbing portion from the nasal cavity thereby the hand operating the holding portion.

9. The operating method of the pharmaceuticals carrier as claimed in claim 8,
wherein the step of the absorbing portion which is soaked is put into the nasal cavity further comprises a step:
the holding portion is operated via the hand, resulting in the absorbing portion in the nasal cavity contacting wall of the nasal cavity.

10. The operating method of the pharmaceuticals carrier as claimed in claim 9,
wherein the medicament which is spilled over from the wall of the nasal cavity is guided to the channel, and the medicament is sustained in the channel after the absorbing portion has been contacted the wall of the nasal cavity.
